(19) United States
(12) Patent Application Publication
(10) Pub. No.: US 2014/0293007 A1
(43) Pub. Date: Oct. 2, 2014

(54) METHOD AND IMAGE ACQUISITION SYSTEM FOR RENDERING STEREOSCOPIC IMAGES FROM MONOSCOPIC IMAGES

(71) Applicants: Ludovic Angot, Hsinchu City (TW); Chun-Te Wu, Taoyuan County (TW); Wei-Jia Huang, Nantou County (TW)

(72) Inventors: Ludovic Angot, Hsinchu City (TW); Chun-Te Wu, Taoyuan County (TW); Wei-Jia Huang, Nantou County (TW)

(73) Assignee: INDUSTRIAL TECHNOLOGY RESEARCH INSTITUTE, Hsinchu (TW)

(21) Appl. No.: 14/356,885

(22) PCT Filed: Nov. 8, 2011
(86) PCT No.: PCT/CN2011/081919

§ 371 Date: May 8, 2014

(51) Int. Cl. A61B 1/00

(52) U.S. Cl. CPC A61B 1/00193
USPC 348/45

(57) ABSTRACT

A method and an image acquisition system for rendering stereoscopic images from monoscopic images are provided. In the present method, an imaging unit of the image acquisition system is moved laterally back and forth to capture a plurality of images. Then, a disparity between each two of the captured images is computed and a one or more pairs of images having an appropriate fixed disparity is selected from the captured images. Finally, the selected one or more pairs of images is displayed so as to render a stereoscopic image.

### BACKGROUND

1. Field of the Disclosure

The disclosure relates to a method and an image acquisition system for rendering stereoscopic images from monoscopic images.

2. Description of Related Art

Minimal Invasive Surgery (MIS) uses an imaging unit and instruments such as graspers, all of small diameters in order to reduce the sequels of the surgical intervention. The imaging unit is in most of the cases a monoscopic endoscope consisting of an optical system and a sensor or the like, associated to a display for the surgeon to observe the operating field. The monoscopic nature of the imaging unit imposes on surgeons a long and tedious training period prior to be able to operate without having the sensation of depth.

Once the surgeon has acquired the skills to perform operations with an endoscope, the operating time remains relatively long due to the added difficulty brought by the limited depth sensation. One solution is to provide the surgeon with depth sensation though a stereoscopic endoscope, but such a device is not only costly, it is also bulkier and offers a limited angular field of view compared to the widely available monoscopic endoscopes. Therefore, there is a need to provide stereoscopic images from monoscopic images captured by monoscopic endoscopes. However, obtaining stereoscopic images from a series of monoscopic images usually suffers from poor results, and there is therefore the need to provide stereoscopic images from monoscopic images with accurate stereoscopic effect.

### SUMMARY OF THE DISCLOSURE

The disclosure is directed to a method and an image acquisition system for rendering stereoscopic images from monoscopic images, in which said monoscopic images with a fixed disparity are appropriately selected to form stereoscopic images.

The disclosure provides a method for rendering stereoscopic images from monoscopic images, adapted to an image acquisition system having an imaging unit. In the method, the imaging unit is moved laterally and a plurality of images is captured. A disparity between pairs of the captured images is computed and one or more pairs of images having an appropriate fixed disparity are selected from the captured images. Finally, the selected pairs of images are displayed in order to render stereoscopic images.

The disclosure provides an image acquisition system, which comprises an imaging unit having a lens and an image sensor, a processing unit, and a display unit. The processing unit is coupled to the image sensor and configured to receive a plurality of images captured by the imaging unit, compute a disparity between pairs of the captured images, and select from the one or more pairs of images having an appropriate fixed disparity. The display unit is coupled to the processing unit and configured to display the pairs of images selected by the processing unit to render stereoscopic images.

In order to make the aforementioned and other features and advantages of the disclosure comprehensible, several exemplary embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a flowchart illustrating a method for rendering stereoscopic images from monoscopic images according to the first embodiment of the disclosure.

FIG. 2 is a schematic diagram illustrating the movement and the disposition of the imaging unit.

FIG. 3A and FIG. 3B are block diagrams of an image acquisition system according to the first embodiment of the disclosure.

FIG. 4A and FIG. 4B are block diagrams of an image acquisition system according to the second embodiment of the disclosure.

FIG. 5 is a flowchart illustrating a method for rendering stereoscopic images from monoscopic images according to the second embodiment of the disclosure.

FIG. 6 is an example of obtaining positions of the imaging unit according to the second embodiment of the disclosure.

FIG. 7 is a block diagram of the processing unit 14 in FIG. 1.

FIG. 8 is a flowchart illustrating a method for rendering stereoscopic images from monoscopic images according to the third embodiment of the disclosure.

FIG. 9 is an example for computing motion vectors between consecutive images according to the third embodiment of the disclosure.

FIG. 10 is an example of image correction for view perspective.

FIG. 11 is an example of image correction for vertical disparity.

FIG. 12(a) and FIG. 12(b) are examples of selecting regions of interest.

FIG. 13(a) and FIG. 13(b) are examples of selecting stereo pairs.

FIG. 14 is an example of the data structure for storing the images.

### DESCRIPTION OF THE EMBODIMENTS

The disclosure makes use of computer vision techniques, position sensors and image processing techniques to select images with a fixed disparity to form one or more stereo pairs of images, such that the user of the system does not suffer from watching stereoscopic images with varying stereo effects.

### First Embodiment

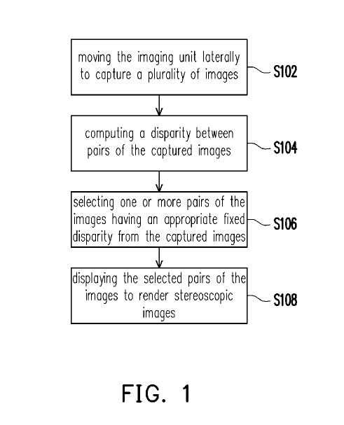

FIG. 1 is a flowchart illustrating a method for rendering stereoscopic images from monoscopic images according to the first embodiment of the disclosure. Referring to FIG. 1, the present method is adapted to an image acquisition system having an imaging unit. Below, various steps of the method provided by the disclosure will be described.

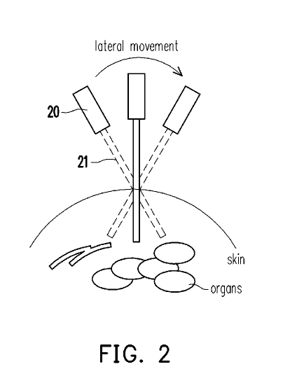

First, the imaging unit is moved laterally back and forth so as to capture a plurality of images (step S102). For example, FIG. 2 is a schematic diagram illustrating the movement and the disposition of the imaging unit. Referring to FIG. 2, the imaging unit 20 is, for example, inserted in a cavity through a trocar 21 inserting in the skin of a patient. The surgeon or the operator moves laterally back and forth the imaging unit 20 so as to capture a plurality of images of the organs inside the cavity from different viewing angles.

Next, a disparity between each two of the captured images is computed (step S104). In detail, the key aspect of the disclosure is to select images with appropriate fixed disparity so as to render stereoscopic images not only with good stereo quality, but with a consistent stereoscopic effect. The disparity may be computed through two methods. One method is to detect the position of the imaging unit thanks to a position sensor and then the detected positions are used to compute the disparity between each pairs of the captured images. The other method is to compute motion vectors of particular features between a N^{th} capture image and each of the M previously captured images, in which M and N are positive integers, and then the computed motion vectors are used to compute the disparity between each pairs of the captured images. Detailed content of the aforesaid two methods will be described below with respective embodiments.

Back to FIG. 1, after the computation of disparity is completed, one or more pairs of images having an appropriate fixed disparity are selected from the captured images (step S106). In detail, the computed disparity may be compared with a predetermined disparity range so as to determine whether the computed disparity is within an appropriate range. Once the disparity between two images is determined as being within the predetermined disparity range, the two images are determined as having appropriate fixed disparity, and therefore can be selected to form one or more pairs of stereoscopic images which are rendered on the appropriate display.

Finally, the selected one or more pairs of images are outputted for display, so as to render stereoscopic images for the operator (step S108). Since the displayed one or more pairs of images has appropriate fixed disparity, the stereoscopic images rendered may give appropriate sensation of depth for the surgeon or the operator using the image acquisition system.

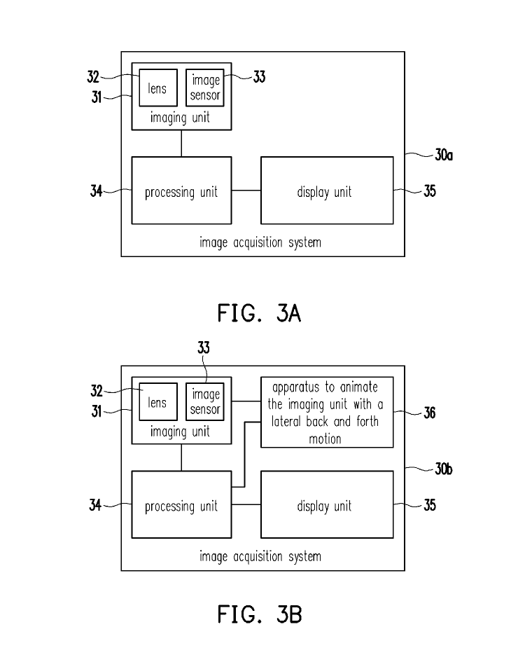

FIG. 3A and FIG. 3B are block diagrams of an image acquisition system according to the first embodiment of the disclosure. Referring to FIG. 3A, the image acquisition system 30a is, for example, an endoscope, a borescope, or any other kind of scope, which comprises an imaging unit 31 having a lens 32 and an image sensor 33, a processing unit 34, and a display unit 35. Referring to FIG. 3B, the image acquisition system 30b further comprises an apparatus 36 which can be in a form of a robotic arm or other mechanical or electromechanical apparatus to animate the imaging unit 31 (or a number of imaging units) with a lateral back and forth movement.

The lens 32 consists of a plurality of optical elements and is used to focus on a target to be captured. The image sensor 33 is, for example, a charge coupled device (CCD) or a complementary metal-oxide-semiconductor (CMOS) sensor disposed after the lens 32 and is used for capturing images. The apparatus 36 is, for example, a robotic arm, or a human operator using the system 30b of the disclosure.

The processing unit 34 is, for example, a central processing unit (CPU), a programmable microprocessor, a digital signal processor (DSP), a programmable controller, an application specific integrated circuit (ASIC), a programmable logic device (PLD), or any other similar device. The processing unit 34 is coupled to the imaging unit 31 so as to receive and process the images captured by the imaging unit 31.

The display unit 35 is, a liquid crystal display (LCD), a plasma display, or a light-emitting diode (LED) display capable of displaying stereoscopic images. The display unit 35 is coupled to the processing unit 34 for displaying the images selected by the processing unit 34 so as to render one or more stereoscopic images.

The image acquisition system 30a or 30b may be used to render stereoscopic images from monoscopic images according to the method illustrated in FIG. 1. Below, various steps of the method provided by the disclosure will be described with reference to various components in the image acquisition system 30b.

First, the imaging unit 31 of the image acquisition system 30b is moved laterally back and forth by the apparatus 36 so as to capture a plurality of images. Next, the processing unit 34 computes a disparity between pairs of the captured images. After the computation of disparity is completed, the processing unit 34 selects one or more pairs of images having an appropriate fixed disparity from the captured images. Finally, the processing unit 34 outputs the selected one or more pairs of images to the display unit 35 for display, so as to render stereoscopic images for the operator.

### Second Embodiment

In this embodiment, positions of imaging unit are successively detected and used for computing disparities between images captured by the imaging unit, so as to select the images suitable for rendering stereoscopic images.

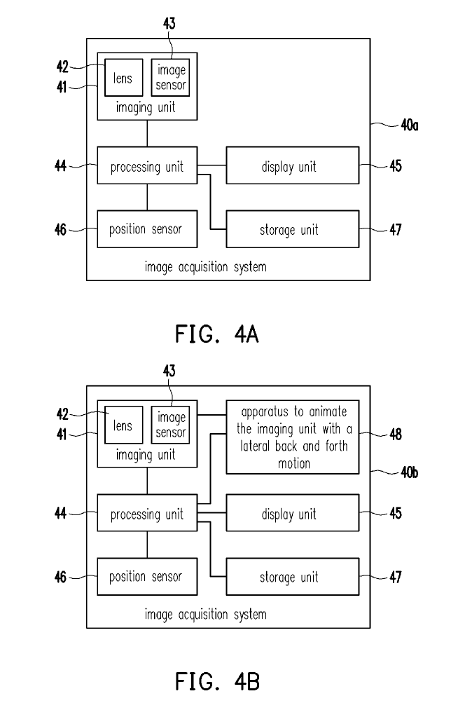

FIG. 4A and FIG. 4B are block diagrams of an image acquisition system according to the second embodiment of the disclosure. Referring to FIG. 4A, the image acquisition system 40a comprises an imaging unit 41 having a lens 42 and an image sensor 43, a processing unit 44, a display unit 45, a position sensor 46, and a storage unit 47. The lens 42 and the image sensor 44 form the imaging unit 41, which is for example an endoscope, a borescope or any kind of scope. Referring to FIG. 4B, the image acquisition system 40b further comprises an apparatus 48 which can be in a form of a robotic arm or other mechanical or electromechanical apparatus to animate the imaging unit 41 with a lateral back and forth movement. Functions of the lens 42, the image sensor 43, the apparatus 48, and the display unit 45 are the same as or similar to those of the lens 32, the image sensor 33, the apparatus 36 and the display unit 35 in the first embodiment, thus the detailed description is not repeated herein.

The position sensor 46 is, for example, a magnetic sensor, an electro-magnetic sensor, an optical sensor, an ultrasound sensor, a radio-frequency sensor, or any other kind of sensor, which is not limited thereto. The position sensor 46 is used to detect a plurality of positions of the imaging unit 41 moving laterally.

The storage unit 47 is, for example, a hard disk or a memory, which is configured to store the images captured by the imaging unit 41 and store the disparities computed by the processing unit 44, so as to be retrieved by the processing unit 44 to select the one or more pairs of images having the appropriate fixed disparity and display the selected one or more pairs of images.

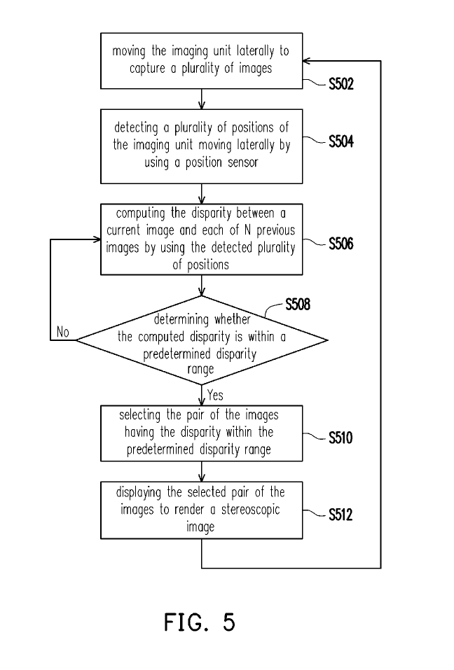

FIG. 5 is a flowchart illustrating a method for rendering stereoscopic images from monoscopic images according to the second embodiment of the disclosure. Referring to FIG. 5, the present method is adapted to the image acquisition system 40b illustrated in FIG. 4B. Below, various steps of the method provided by the disclosure will be described with reference to various components in the image acquisition system 40b.

First, the imaging unit 41 is moved laterally by the apparatus 48 or by a human operator so as to capture a plurality of images (step S502). Next, the position sensor 46 is used to detect a plurality of positions of the imaging unit 41 moving laterally (step S504).

Then, the disparity between the N^{th} capture image and each of the M previous captured images is computed by using the plurality of positions detected by the position sensor 46 (step S506), in which M and N are positive integers. In detail, the disparity is obtained by deducing the lateral movement of the image based on the coordinates detected by the position sensor 46. Typically, the position sensor 46 can provide 6 coordinates. That is, x, y, z, pitch, roll, and yaw. Based on the intrinsic and extrinsic parameters of the imaging unit 41 and the location where the position sensor 46 is disposed on the imaging unit 41, the disparity between images can be deduced.

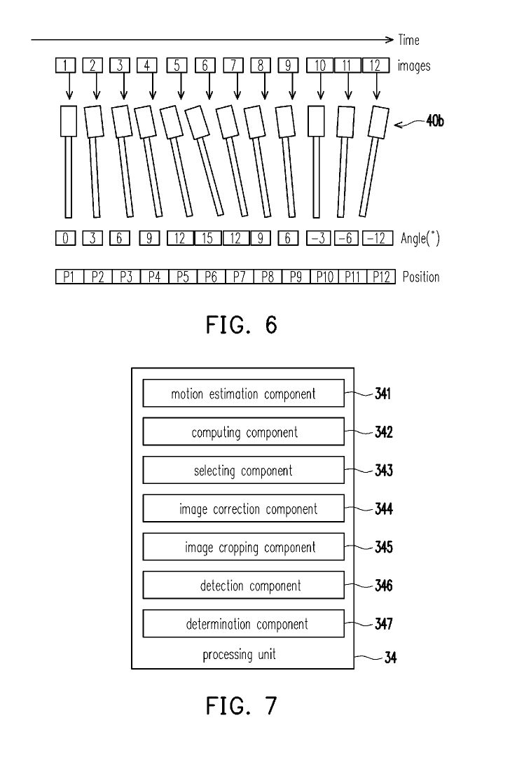

For example, FIG. 6 is an example of obtaining positions of the imaging unit according to the second embodiment of the disclosure. FIG. 6 shows the positions of the imaging unit 41 at different instants, during which the imaging unit gradually moves to the left side, moves to a vertical position, and then moves to the right side. In this illustrative example, twelve images are successively captured by the imaging unit 41, and the coordinates of the imaging unit are also detected, so as to be used to compute the disparity between the captured images.

Referring back to FIG. 5, after the computation of the disparity is completed, the processing unit 44 compares the computed disparity with a predetermined disparity range so as to determine whether the computed disparity between pairs of captured images is within a predetermined disparity range (step S508). The predetermined disparity range may comprise a horizontal disparity range and a vertical disparity range and the one or more pairs of images is determined to be appropriate to render a stereoscopic image only when the horizontal disparity disp_x and the vertical disparity disp_y thereof satisfy following conditions:

dxₘᵢₙ<dispₓ<dxₘₐₓ ; and

0<disp_{y}<dyₘₐₓ.

The dxₘᵢₙ and dxₘₐₓ respectively represent a minimum and a maximum of the horizontal disparity range, and dyₘₐₓ represents a maximum of the vertical disparity range. Indeed, the lateral movement of the imaging unit may not be strictly corresponding to a horizontal motion, and therefore the parameter dyₘₐₓ represents the maximum acceptable vertical movement for the imaging unit. The aforesaid limits of disparity range may be obtained based on the resolution of the image sensor and the resolution of the display unit, and can be obtained also by taking into account the extrinsic characteristics of the imaging unit such as the magnification ratio, or a distance between a reference point in the imaging unit and an object under observation, and by taking into account the extrinsic characteristics of a stereoscopic display system that displays the selected pairs of images such as a viewing distance and a size of the display.

Once the disparity between two images is determined as being within the predetermined disparity range, the two images are determined as having appropriate fixed disparity, and accordingly processing unit 44 may select from the captured images, the pair of images having the disparity within the predetermined disparity range (step S510).

Finally, the processing unit 44 outputs the selected one or more pairs of images to the display unit 45 and then the display unit 45 displays the selected one or more pairs of images to render the one or more stereoscopic image (step S512). After the display of stereoscopic images, the flow is returned to step S502, so as to continuously search for pairs of image to be displayed.

It is noted herein that, in the present embodiment, the one or more pairs of images having the appropriate fixed disparity are selected and displayed right after the disparities are computed. However, in another embodiment, the captured images and the computed disparities may be stored in the storage unit 47. When the surgeon or the operator of the image acquisition system 40b needs to see the stereoscopic images, he/she may activate the 3D view function. Meanwhile, the image acquisition system 40b receives a request for the 3D view and accordingly retrieves the closest in time previously stored images and disparities so as to select the one or more pairs of images having the appropriate fixed disparity and display the selected one or more pairs of images. It is to be understood that the time delay between a request for a 3D view and the actual display can be very short so as to be unnoticeable by the operator.

### Third Embodiment

In this embodiment, motion vectors of particular features between each two of images captured by the imaging unit are computed and used for computing the disparities between the images, so as to select the one or more pairs of images suitable for obtaining one or more stereoscopic images.

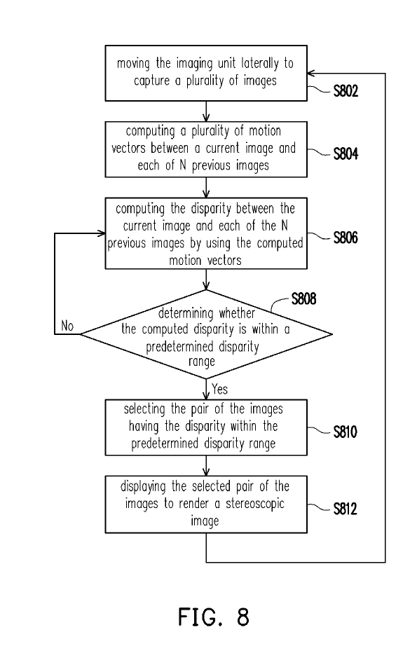

FIG. 7 is a block diagram of the processing unit 34 in FIG. 3B. FIG. 8 is a flowchart illustrating a method for rendering stereoscopic images from monoscopic images according to the third embodiment of the disclosure. Referring to FIG. 7, the processing unit 34 comprises a motion estimation component 341, a computing component 342, a selecting component 343, an image correction component 344, an image cropping component 345, a detection component 346, and a determination component 347. Referring to FIG. 8, the present method is adapted to the image acquisition system 30b illustrated in FIG. 3B and the processing unit 34 illustrated in FIG. 7. Below, various steps of the method provided by the disclosure will be described with reference to various components in the image acquisition system 30b.

First, the imaging unit 31 is moved laterally back and forth by the apparatus 36 or by a human operator so as to capture a plurality of images (step S802). Next, the motion estimation component 341 of the processing unit 34 computes a plurality of motion vectors between a N^{th} capture image and each of the M previous captured images (step S804), in which M and N are positive integers. In detail, a plurality of feature points are tracked in consecutive images captured by the image sensor 33 and the motion vectors of these feature points are computed by using computer vision methods, for example, Lukas Kanade tracking algorithm.

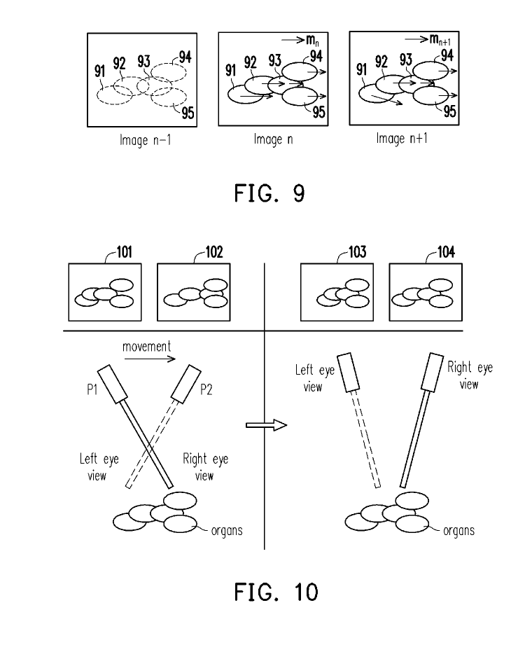

FIG. 9 is an example for computing motion vectors between consecutive images according to the third embodiment of the disclosure. Referring to FIG. 9, three consecutive images comprising image n−1, image n, and image n+1 are given, in which each of the image n−1, image n, and image n+1 comprises the same features, which are organs 91˜95. The motion vectors of organs 91˜95 between image n−1 and image n are computed and averaged into an average motion vector mₙ. The motion vectors of organs 91˜95 between image n and image n+1 are computed and summed to form an average motion vector mₙ₊₁. The computed motions vectors mₙ and mₙ₊₁ provides a direct relationship to the disparities between the images n−1, image n, and image n+1 providing that the object under observation are immobile or animated by a slow motion compared to the lateral motion of the imaging unit.

Referring back to FIG. 8, the computing component 342 of the processing unit 34 computes the disparity between the N^{th} capture image and each of the M previous captured images by using the motion vectors computed by the motion estimation component 341 (step S806).

After the computation of the disparity is completed, the selecting component 343 of the processing unit 34 compares the computed disparity with a predetermined disparity range so as to determine whether the computed disparity between pairs of captured images is within a predetermined disparity range (step S808).

Once the disparity between two images is determined as being within the predetermined disparity range, the two images are determined as having an appropriate fixed disparity, and accordingly the selecting component 343 may select the one or more pairs of images having the disparity within the predetermined disparity range from the captured images (step S810).

Finally, the selecting component 343 outputs the selected one or more pairs of images to the display unit 35 and then the display unit 35 displays the selected one or more pairs of images to render the one or more stereoscopic images (step S812). After the display of stereoscopic images, the flow is returned to step S802, so as to continuously search for pairs of image to be displayed.

It is noted herein that the present embodiment provides several methods for correcting images in accordance with various distortion found while capturing images, so as to render stereoscopic images with fine quality.

FIG. 10 is an example of image correction for view perspective. Referring to FIG. 10, when the imaging unit is at positioned P1, the image 101 captured thereby has a distortion corresponding to an observation angle slightly shifted to the right of organs compared to the actual left eye view of the user. Similarly, when the imaging unit is at positioned P2, the image 102 captured thereby has a distortion corresponding to an observation angle slightly shifted to the left of organs compared to the actual right eye view of the user. To correct the aforesaid distortion, the image correction component 344 of the processing unit 34 applies an image correction to the selected one or more pairs of images 101 and 102 to rectify a viewing angle of the imaging unit to fit the viewing angle of a human eye. To be specific, the image 101 captured by the imaging unit at position P1 is corrected to be the image 104 of the right eye view and the image 102 captured by the imaging unit at position P2 is corrected to be the image 103 of the left eye view. Accordingly, the one or more pairs of images 101 and 102 can be seen in correct view perspective by the user.

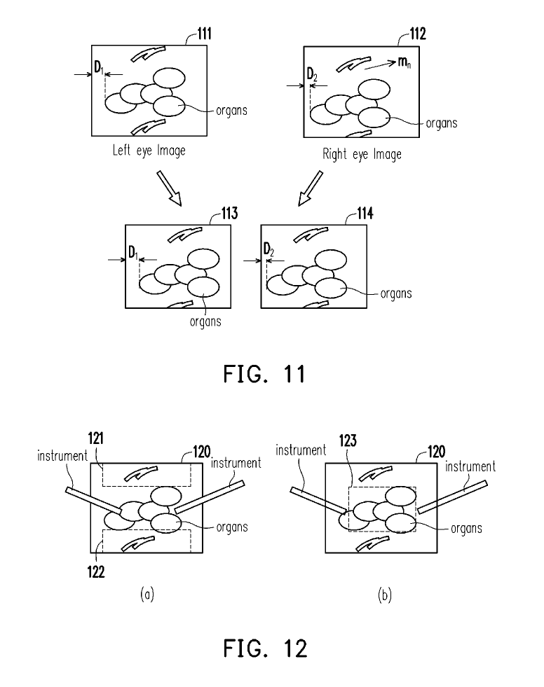

FIG. 11 is an example of image correction for vertical disparity. Referring to FIG. 11, image 111 is captured as a left eye image in which the left edge of organs in image 111 has a distance D1 away from the left end of image 111. The image 112 is captured as a right eye image in which the left edge of organs in image 112 has a distance D2 away from the left end of image 112. In addition to the horizontal disparity between images 111 and 112, it is noted that there is also a vertical disparity between images 111 and 112, which causes the image 112 to correspond to a point of view slightly above that of the image 111. To correct the aforesaid distortion caused by the vertical disparity, the image cropping component 345 of the processing unit 34 crops the images 111 and 112 so that no vertical disparity exists between both images of each of the selected pairs of images, so that each one or more pairs of images can be merged by a human viewer into a comfortable stereoscopic image. As shown in FIG. 11, the upper portion of image 111 is cropped to render the image 113 and the lower portion of image 112 is cropped to render the image 114. Through the cropping, the vertical disparity between images 111 and 112 is eliminated, and the cropped images 113 and 114 can be used to render a stereoscopic image with appropriate disparity.

Further, it is noted that in the images captured by the imaging unit, some objects such as instruments of operation may move themselves while the imaging unit moves, and the movement may cause an uncomfortable feeling for the user. To minimize the influence of the movement of the objects in the images, at least one region of interest (ROI) is chosen for subsequent processing. That ROI allows evaluating the motion of objects such as graspers in the field of view of the imaging unit, in order to eliminate one or more pairs of images where the movement in one frame is different from that of the other frame forming a pair of image with the correct fixed disparity. FIG. 12(a) and FIG. 12(b) are examples of selecting regions of interest. Referring to FIG. 12(a) and FIG. 12(b), image 120 is the image originally captured by the imaging unit and which comprises a region of organ and some regions involving instruments. To eliminate one or more pairs of images where the movement in one frame is different from that of the other frame forming a pair of image with the correct fixed disparity, the detection component 346 of the processing unit 34 detects at least one moving object in the captured image 120 and the determination component 347 rejects the pair of images in which one of the image contains a motion which is different from that of the other image. In FIG. 12(a), a region 121 in the upper portion of image 120 and a region 122 in the lower portion of image 120 are determined as the regions of interest and used for computing the motion vectors. In FIG. 12(b), a region 123 in the central portion of image 120 is determined as the region of interest and used for computing the motion vectors.

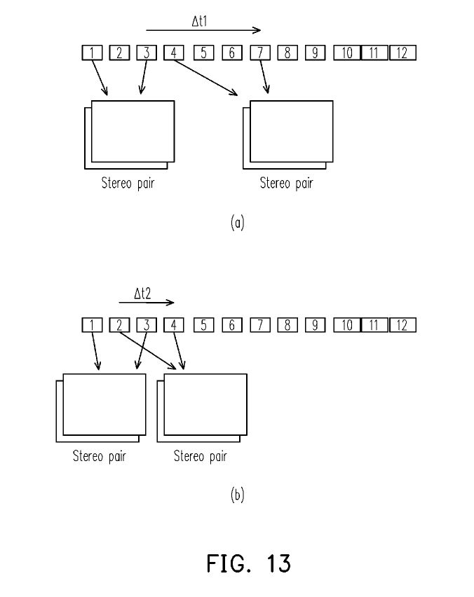

To select multiple pairs of images, the present disclosure provides two scenarios according to different requirements of the user. FIG. 13(a) and FIG. 13(b) are examples of selecting stereo pairs. Referring to FIG. 13(a) as an illustrative example, in the first scenario, image 1 and image 3 are selected as a first stereo pair since the disparity there between is determined as within the appropriate disparity range. To select another one or more pairs of images, the image acquisition system checks the disparity between the image next to image 3 (i.e. the image 4) and each of the images after image 4, and finally selects image 4 and image 7 as a next one or more pairs of images having the appropriate fixed disparity. Referring to FIG. 13(b) as an illustrative example for the second scenario, image 1 and image 3 are also selected as a first stereo pair of images. In order to select another pair of images, the image acquisition system checks the disparity between the image next to image 1 (i.e. the image 2) and each of the images after image 2, and finally selects image 2 and image 4 as a next one or more pairs of images having the appropriate fixed disparity. The time delay Δt1 between the selection of 2 consecutive pairs of images in the first scenario is longer than the time delay Δt2 between the selection of 2 consecutive pairs of images in the second scenario. The second scenario is more suitable for displaying stereoscopic images with a higher rate compared to that of the first scenario. However, the load for computing disparities in the second scenario is higher than that in the first scenario, such that the second scenario may require a processor with a higher computing power.

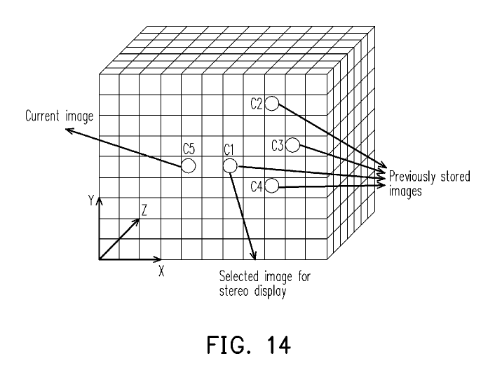

Finally, the disclosure introduces a data structure for storing the images captured by the imaging unit. FIG. 14 is an example of the data structure for storing the images. Referring to FIG. 14, the 3D space is divided into a plurality of cells, and each cell is used to store the image captured at the corresponding position where the imaging unit is detected by the position sensor. As shown in FIG. 14, cells C1 to C4 are used to stored image data of the images previously captured by the imaging unit. When a current image is captured at a position corresponding to cell C5, the image data of the current image is stored in the cell C5, and the position of cell C5 is compared with the positions of cells C1 to C4, so as to find the image having an appropriate fixed disparity with the current image. If the appropriate fixed disparity is set as a width of two cells, then the image with data stored in cell C1 is considered as a suitable image to render a stereoscopic image with the current image whose data is stored in cell C5.

In summary, the method and the image acquisition system for rendering stereoscopic images for rendering stereoscopic images form monoscopic images of the disclosure select pairs of images with appropriate fixed disparity so as to render stereoscopic images with a stereoscopic effect closer to a stereoscopic image acquisition system compared to that of most of the 2D to 3D conversion algorithms. Accordingly, the disclosure may provide a surgeon or other operators with a depth sensation of the operating field when the operation is performed in a restricted space. As a result, the surgeon or operators may visually be assisted with a depth perception of an operation field in order to better position his/her instruments with respect to the organs, therefore facilitating the operation and reducing the time of operation.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. A method for rendering stereoscopic images from monoscopic images, adapted to an image acquisition system having an imaging unit, the method comprising:
moving the imaging unit laterally to capture a plurality of images;
computing a disparity between pairs of the captured images;
selecting one or more pairs of the images having an appropriate fixed disparity from the plurality of captured images; and
displaying the selected pairs of images to render stereoscopic images.

2. The method for rendering stereoscopic images from monoscopic images as claimed in claim 1, wherein the step of computing the disparity between pairs of captured images comprises:
computing a plurality of motion vectors between a N^{th} captured image and each of M previously captured images, wherein M and N are positive integers;
computing the disparity between the N^{th} captured image and each of the M previously captured images by using the computed motion vectors.

3. The method for rendering stereoscopic images from monoscopic images as claimed in claim 2, wherein the motion vectors are computed in a plurality of regions of interests of the images, and the plurality of regions of interests are chosen.

4. The method for rendering stereoscopic images from monoscopic images as claimed in claim 1, wherein the step of computing the disparity between pairs of images comprises:
detecting a plurality of positions of the imaging unit moving laterally by using a position sensor disposed on the imaging unit or installed inside the imaging unit; and
computing the disparity between the N^{th} captured image and each of the M previously captured images by using the detected plurality of positions, wherein M and N are positive integers.

5. The method for rendering stereoscopic images from monoscopic images as claimed in claim 4, wherein the position sensor utilizes either one or a combination of the following technology: magnetic, electro-magnetic, optical, ultrasound, and radio-frequency.

6. The method for rendering stereoscopic images from monoscopic images as claimed in claim 1, wherein the step of selecting the one or more pairs of images having an appropriate fixed disparity from the plurality of captured images comprises:
determining whether the computed disparity between the pairs of captured images is within a predetermined disparity range; and
selecting the one or more pairs of images having the disparity within the predetermined disparity range from the plurality of captured images.

7. The method for rendering stereoscopic images from monoscopic images as claimed in claim 1, wherein after the step of selecting the one or more pairs of images having an appropriate fixed disparity from the plurality of captured images, the method further comprises:
deducing the appropriate fixed disparity from a plurality of characteristics of the imaging unit, which comprise a magnification ratio of an optical system, a distance between a reference point in the imaging unit and an object under observation; and
deducing the appropriate fixed disparity from a plurality of characteristics of a stereoscopic display system, which comprises a viewing distance and a size of the display.

8. The method for rendering stereoscopic images from monoscopic images as claimed in claim 1, wherein after the step of computing the disparity between pairs of the captured images, the method further comprises:
storing the captured images and the disparity between the pairs of captured images; and
retrieving the stored images and disparities;
selecting the one or more pairs of images having the appropriate fixed disparity; and
displaying the selected one or more pairs of images for a 3D view.

9. The method for rendering stereoscopic images from monoscopic images as claimed in claim 1, wherein after the step of selecting the one or more pairs of images having an appropriate fixed disparity from the plurality of captured images, the method further comprises:
applying an image correction to the selected one or more pairs of images to rectify a viewing angle of the imaging unit to fit the viewing angle of a human eye.

10. The method for rendering stereoscopic images from monoscopic images as claimed in claim 1, wherein after the step of selecting the one or more pairs of images having the appropriate fixed disparity from the plurality of captured images, the method further comprises:
cropping vertically one or both images of each of the selected pairs of images.

11. The method for rendering stereoscopic images from monoscopic images as claimed in claim 1, wherein before the step of computing the disparity between pairs of the captured images, the method further comprises:
determining a region of interest within the captured images where an object penetrates in a field of view of the captured image; wherein
the determined region of interest within the captured images is used to compute the disparity.

12. The method for rendering stereoscopic images from monoscopic images as claimed in claim 1, wherein before the step of computing the disparity between pairs of the captured images, the method further comprises:
rejecting a pair of frames having the appropriate fixed disparity, if an object moves differently in one frame compared to the other.

13. The method for rendering stereoscopic images from monoscopic images as claimed in claim 1, wherein after the step of selecting the one or more pairs of images having the appropriate fixed disparity from the captured images, the method further comprises:
selecting an another one or more pairs of the images having the appropriate fixed disparity from the images starting from the image next to a first image or a second image of the previously selected one or more pairs of images.

14. An image acquisition system, comprising:
an imaging unit comprising a lens; and
an image sensor;
a processing unit, coupled to the image sensor and configured to receive a plurality of images captured by the imaging unit, compute a disparity between pairs of the captured images, and select the one or more pairs of images having an appropriate fixed disparity from the plurality of captured images; and
a display unit, coupled to the processing unit and configured to display the pairs of images selected by the processing unit to render stereoscopic images.

15. The image acquisition system as claimed in claim 14, further comprising:
an apparatus animate the imaging unit with a lateral back and forth motion.

16. The image acquisition system as claimed in claim 14, wherein the processing unit comprises:
a motion estimation component, configured to compute a plurality of motion vectors between a N^{th} captured image and each of M previously captured images, wherein M and N are positive integers; and
a first computing component, configured to compute the disparity between the N^{th} image and each of the M previously captured images by using the computed motion vectors.

17. The image acquisition system as claimed in claim 14, further comprises:
a position sensor, configured to detect the positions of the imaging unit.

18. The image acquisition system as claimed in claim 17, wherein the processing unit further comprises computing the disparity between a N^{th} captured image and each of the M previously captured images by using the plurality of positions detected by the position sensor, wherein M and N are positive integers.

19. The image acquisition system as claimed in claim 17, wherein the position sensor comprises a magnetic sensor, an optical sensor, an electro-magnetic sensor, a radio-frequency sensor or an ultrasound sensor.

20. The image acquisition system as claimed in claim 14, wherein the processing unit comprises:
a selecting component, configured to determine whether the computed disparity between the pairs of captured images is within a predetermined disparity range, and selecting the one or more pairs of images having the disparity within the predetermined disparity range from the captured plurality of images.

21. The image acquisition system as claimed in claim 14, further comprising:
a storage unit, configured to store the images captured by the imaging unit and the disparity between the pairs of captured images computed by the processing unit.

22. The image acquisition system as claimed in claim 14, wherein the processing unit comprises:
an image correction component, configured to apply an image correction to the selected one or more pairs of images to rectify a viewing angle of the imaging unit.

23. The image acquisition system as claimed in claim 14, wherein the processing unit comprises:
an image cropping component, configured to crop vertically one or both images of each of the selected pairs of images, and each of the selected pairs of images formed stereoscopic image.

24. The image acquisition system as claimed in claim 14, wherein the processing unit comprises:
a detection component, configured to detect at least one moving object in the captured images; and
a determination component, configured to determine a region of interest of the captured images to exclude the region comprising the at least one moving object, wherein
the captured images within the determined region of interest are used to compute the disparity.
